(19) United States
(12) Patent Application Publication
(10) Pub. No.: US 2026/0166298 A1
(43) Pub. Date: Jun. 18, 2026

(54) PERCUTANEOUS CIRCULATORY SUPPORT DEVICE WITH BRAIDED DISTAL TIP

(71) Applicant: BOSTON SCIENTIFIC SCIMED, INC., Maple Grove, MN (US)

(72) Inventors: Brice Lee Shireman, Maple Grove, MN (US); Reggie Roth, Monticello, MN (US); Michael Kaland, White Bear Lake, MN (US); Timothy Lawrence Rubesch, Blaine, MN (US)

(73) Assignee: BOSTON SCIENTIFIC SCIMED, INC., Maple Grove, MN (US)

(21) Appl. No.: 19/422,845

(22) Filed: Dec. 17, 2025

Related U.S. Application Data
(60) Provisional application No. 63/735,374, filed on Dec. 18, 2024.

(51) Int. Cl. A61M 60/13; A61M 25/00; A61M 60/216; A61M 60/416

(52) U.S. Cl. CPC A61M 60/13; A61M 60/216; A61M 60/416; A61M 25/0053; A61M 25/0069; A61M 25/0074

(57) ABSTRACT

A percutaneous circulatory support device including a blood pump and a distal tip portion coupled to the blood pump. The distal tip portion includes of a multi-layered structure, with an integrated braided reinforcement layer. The braided reinforcement layer includes a tubular braid, wherein the braid has a varying stiffness from a proximal end of the braid to a distal end of the braid.

### CROSS REFERENCE TO RELATED APPLICATIONS

This application claims the benefit of U.S. Provisional Patent Application Ser. No. 63/735,374 filed on Dec. 18, 2024, the disclosure of which is incorporated herein by reference.

### TECHNICAL FIELD

The present disclosure generally relates to percutaneous circulatory support systems. More specifically, the disclosure relates to percutaneous circulatory support devices that include a distal tip portion with a braided wire support.

### BACKGROUND

Percutaneous mechanical circulatory support devices, such as blood pumps, can provide transient support for hours or months of use in patients whose heart function or cardiac output is compromised. Issues may complicate delivery of blood pumps within the heart resulting in decreased performance. Accordingly, there is an ongoing need to provide alternative configurations of percutaneous mechanical circulatory support devices to facilitate delivery and use of the percutaneous mechanical circulatory support devices within the heart of a patient.

### BRIEF SUMMARY

A first example is a percutaneous circulatory support device. The device includes a blood pump including an inlet and an outlet; an impeller disposed within a housing of the blood pump and rotatable relative to the housing to cause blood to flow into the inlet, through the housing, and out of the outlet; a motor operably coupled to the impeller and configured to rotate the impeller relative to the housing; and a distal tip portion coupled to the blood pump and extending distally therefrom. The distal tip portion has a layered structure, including an inner layer, an outer layer, and a braided reinforcement layer including a braid disposed between the inner layer and the outer layer.

Alternatively or additionally to any of the examples herein, the braid is embedded within the inner layer.

Alternatively or additionally to any of the examples herein, the braid is embedded within the outer layer.

Alternatively or additionally to any of the examples herein, the braid is a distinct structure positioned in between the outer layer and the inner layer.

Alternatively or additionally to any of the examples herein, the braid comprises a stiffness profile that varies a stiffness of the braid along a length of the distal tip portion.

Alternatively or additionally to any of the examples herein, a distal end of the braid has a first pic count and a proximal end of the braid has a second pic count, wherein the first pic count is different than the second pic count.

Alternatively or additionally to any of the examples herein, the first pic count is greater than the second pic count, and the braid is configured to provide a gradual transition in pic count along the length of the distal tip portion from the distal end of the braid to the proximal end of the braid.

Alternatively or additionally to any of the examples herein, a ratio between the first pic count and the second pic count is 3:1 or more.

Alternatively or additionally to any of the examples herein, the first pic count is in a range of 100 to 200, and the second pic count is in a range of 10 to 30.

Alternatively or additionally to any of the examples herein, the braid comprises a round wire.

Alternatively or additionally to any of the examples herein, the braid comprises a ribbon wire.

Alternatively or additionally to any of the examples herein, the braid comprises a radiopaque material.

Alternatively or additionally to any of the examples herein, the braid comprises a nitinol material.

Alternatively or additionally to any of the examples herein, the braid comprises a mixture of materials.

Alternatively or additionally to any of the examples herein, the distal tip portion has a flared proximal end configured to couple with a distal end of the blood pump.

Alternatively or additionally to any of the examples herein, the distal tip portion is coupled to the distal end of the blood pump via an adapter.

Alternatively or additionally to any of the examples herein, the braid extends along the flared proximal end of the distal tip portion.

Alternatively or additionally to any of the examples herein, the flared proximal end of the distal tip portion extends over and overlaps with the adapter.

Alternatively or additionally to any of the examples herein, the flared proximal end of the distal tip portion surrounds a distally extending post of the adapter.

Alternatively or additionally to any of the examples herein, the distal tip portion comprises a distal terminal region devoid of the braid.

Alternatively or additionally to any of the examples herein, the braid extends from the distal end of the distal tip portion to the proximal end of the distal tip portion.

Another example is a percutaneous circulatory support device. The device includes a blood pump including an inlet and an outlet; an impeller disposed within a housing of the blood pump and rotatable relative to the housing to cause blood to flow into the inlet, through the housing, and out of the outlet; a motor operably coupled to the impeller configured to rotate the impeller relative to the housing; an adapter at a distal end of the blood pump, the adapter including a distally extending post; and a distal tip portion surrounding and coupled to the distally extending post of the adapter. The distal tip portion has a layered structure including an inner layer, an outer layer, and a braided reinforcement layer including a braid disposed between the inner layer and the outer layer. The braid comprises a varying pic count along a length of the braid, with a first pic count at a distal end of the braid and a second pic count at a proximal end of the braid.

Alternatively or additionally to any of the examples herein, the braid is embedded within the inner layer.

Alternatively or additionally to any of the examples herein, the braid is embedded within the outer layer.

Alternatively or additionally to any of the examples herein, the braid is a distinct structure positioned in between the outer layer and the inner layer.

Alternatively or additionally to any of the examples herein, the first pic count is different than the second pic count.

Alternatively or additionally to any of the examples herein, the first pic count is greater than the second pic count, and the braid is configured to provide a gradual transition in pic count along the length of the braid from the distal tip portion from the distal end of the braid to the proximal end of the braid.

Alternatively or additionally to any of the examples herein, the braid comprises a round wire.

Alternatively or additionally to any of the examples herein, the braid comprises a ribbon wire.

Alternatively or additionally to any of the examples herein, the braid comprises a radiopaque material.

Alternatively or additionally to any of the examples herein, the braid comprises a nitinol material.

Alternatively or additionally to any of the examples herein, the braid comprises a mixture of materials.

Alternatively or additionally to any of the examples herein, the distal tip portion has a flared proximal end configured to couple with a distal end of the blood pump.

Alternatively or additionally to any of the examples herein, the distal tip portion is coupled to the distal end of the blood pump via an adapter.

Alternatively or additionally to any of the examples herein, the braid extends along the flared proximal end of the distal tip portion.

Alternatively or additionally to any of the examples herein, the flared proximal end of the distal tip portion surrounds a distally extending post of the adapter.

Alternatively or additionally to any of the examples herein, the distal tip portion comprises a distal terminal region devoid of the braid.

Alternatively or additionally to any of the examples herein, the braid extends from the distal end of the distal tip portion to the proximal end of the distal tip portion.

Another example is a percutaneous circulatory support device. The device includes a blood pump including an inlet and an outlet; an impeller disposed within a housing of the blood pump and rotatable relative to the housing to cause blood to flow into the inlet, through the housing, and out of the outlet; a motor operably coupled to the impeller and configured to rotate the impeller relative to the housing; and a distal tip portion coupled to the blood pump and extending distally therefrom. The distal tip portion has a layered structure including an inner layer, an outer layer, and a braided reinforcement layer disposed between the inner layer and the outer layer. The braided reinforcement layer includes a braid having a varying pic count along a length of the braid, with a first pic count at a distal end of the braid and a second pic count at a proximal end of the braid. The second pic count is less than the first pic count and the braid is configured to provide a gradual transition in pic count along the length of the braid from the distal end of the braid to the proximal end of the braid.

Alternatively or additionally to any of the examples herein, the braid is embedded within the inner layer.

Alternatively or additionally to any of the examples herein, the braid is embedded within the outer layer.

Alternatively or additionally to any of the examples herein, the braid is a distinct structure positioned in between the outer layer and the inner layer.

Alternatively or additionally to any of the examples herein, the braid comprises a round wire.

Alternatively or additionally to any of the examples herein, the braid comprises a ribbon wire.

Alternatively or additionally to any of the examples herein, the braid comprises a radiopaque material.

Alternatively or additionally to any of the examples herein, the braid comprises a nitinol material.

Alternatively or additionally to any of the examples herein, the braid comprises a mixture of materials.

Alternatively or additionally to any of the examples herein, the distal tip portion has a flared proximal end configured to couple with a distal end of the blood pump.

Alternatively or additionally to any of the examples herein, the distal tip portion is coupled to the distal end of the blood pump via an adapter.

Alternatively or additionally to any of the examples herein, the braid extends along the flared proximal end of the distal tip portion.

Alternatively or additionally to any of the examples herein, the flared proximal end of the distal tip portion extends over and overlaps with the adapter.

Alternatively or additionally to any of the examples herein, the distal tip portion comprises a distal terminal region devoid of the braid.

Alternatively or additionally to any of the examples herein, the braid extends from the distal end of the distal tip portion to the proximal end of the distal tip portion.

While multiple embodiments are disclosed, other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE SEVERAL VIEWS OF THE DRAWINGS

FIG. 1 is a perspective view of an illustrative percutaneous circulatory support device, in accordance with embodiments of the subject matter disclosed herein.

FIG. 2 is a detail view of a portion of the illustrative percutaneous circulatory support device of FIG. 1.

FIG. 3 is a side sectional view of a distal tip portion of the percutaneous circulatory support device of FIG. 1.

FIG. 4A is a cross-sectional view of the distal tip portion of an illustrative percutaneous circulatory support device.

FIG. 4B is a detail view of a portion of the illustrative percutaneous circulatory support device of FIG. 4A.

FIG. 5 is a cross-sectional view of the distal tip portion of an illustrative percutaneous circulatory support device.

While the disclosure is amendable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

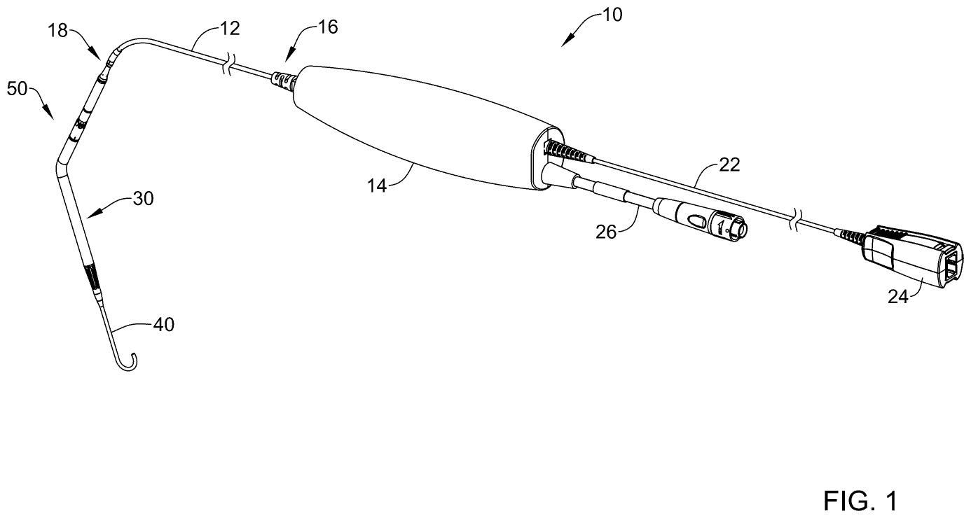

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. For instance, a proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The percutaneous circulatory support device 10 may also include an extension 26 connectable to the controller for sending and/or receiving signals, such as from one or more sensors during operation of the blood pump 50.

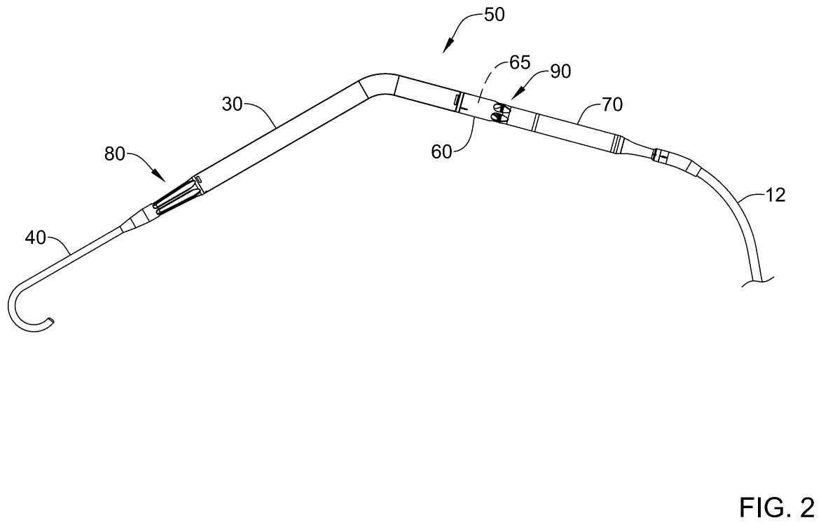

Additional features of the blood pump 50 are illustrated in FIG. 2. FIG. 2 is a detail view of a portion of the illustrative percutaneous circulatory support device of FIG. 1. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller, secured to an impeller shaft, that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

The blood pump 50 may be guided over a guidewire during introduction of the blood pump 50 into the vasculature of a patient. For instance, a guidewire, inserted through a guidewire lumen of the distal tip 40, may be advanced proximally along the impeller assembly 65 and out through one of the outflow windows of the blood outlet 90. With the guidewire tracked through the blood pump 50, the percutaneous circulatory support device 10 may be advanced over the guidewire into a vasculature.

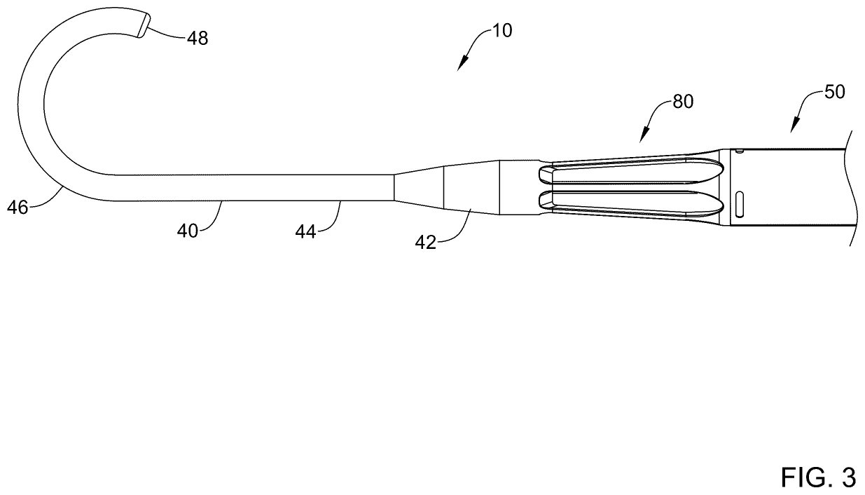

FIG. 3 depicts a side sectional view of a distal tip portion of the percutaneous circulatory support device of FIG. 1. The distal tip portion 40 includes a proximal terminal region 44 adjacent to the blood pump 50 and a distal terminal region 46 extending distal of the proximal terminal region 44 and opposite the blood pump 50. The proximal terminal region 44 may extend proximally to a proximal end 42 of the distal tip portion 40. The distal terminal region 46 may extend distally to the distal end 48 of the distal tip portion 40. In some embodiments and as illustrated, the proximal terminal region 44 has a generally straight shape and the distal terminal region 46 has a generally curved shape. In other embodiments, the proximal terminal region 44 and/or the distal terminal region 46 may have different shapes.

In some embodiments, the proximal terminal region 44 of the distal tip portion 40 is constructed to have a relatively high stiffness compared to the distal terminal region 46 so that it may withstand forces acting on the distal tip portion 40 and the blood pump 50. Such a stiffness also provides axial strength, which facilitates positioning and supporting the cannula in the left ventricle.

In some embodiments, the distal terminal region 46 of the distal tip portion 40 is constructed to have a relatively low flexural stiffness compared to the proximal terminal region 44. In one embodiment, the ratio of flexural stiffness of the proximal terminal region 44 to the distal terminal region 46 is 5:1. In other embodiments, the ratio of flexural stiffness of the proximal terminal region 44 to the distal terminal region 46 may be 3:1 or more, 4:1 or more, 5:1 or more, or 6:1 or more, for example. Such a stiffness facilitates atraumatic contact with tissue yet provides adequate structural strength for positioning and supporting the cannula in the left ventricle while also being capable of absorbing forces acting on the distal tip portion 40.

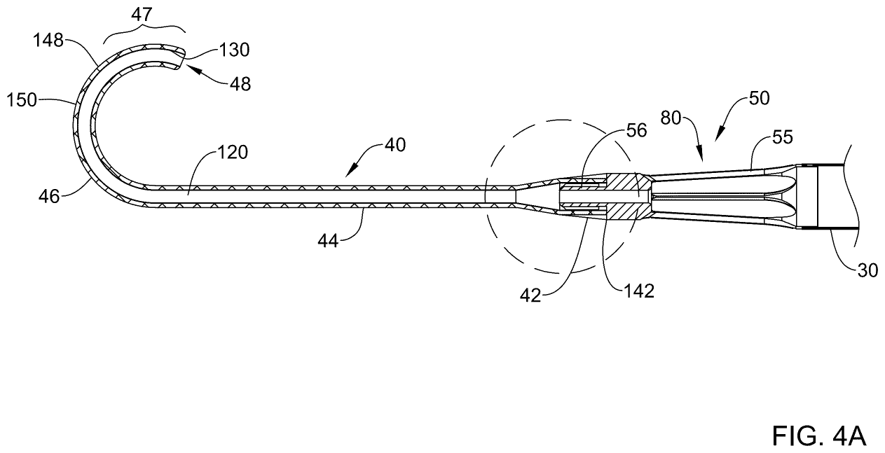

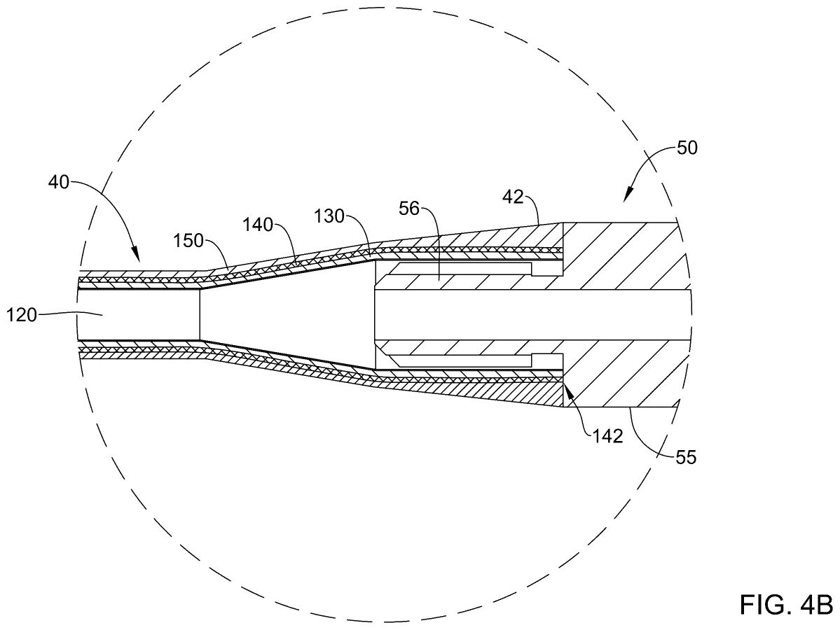

FIG. 4A is a cross-sectional view of the distal tip portion 40 in accordance with embodiments of the subject matter disclosed herein. Specifically, FIG. 4A depicts a view of the distal tip portion 40 attached to a distal end region of the blood pump 50. For example, the proximal terminal region 44 may be secured to an adapter 55 attached to and extending distally from the cannula 30. In some instances, the adapter 55 may define or include one or more blood inflow windows forming the blood inlet 80. In this example, distal tip portion 40, which may be located distally in relation to the blood pump 50, may surround and be coupled to a distally extending post 56 of the adapter 55.

FIG. 4B is an enlarged cross-sectional view depicting the circled region of FIG. 4A, further illustrating aspects of the coupling mechanism between the distal tip portion and the distally extending post 56 of the adapter 55, or other component of the blood pump 50.

In some examples, and as illustrated, the proximal end 42 of the distal tip portion 40 includes a flared or enlarged diameter region which is flared outward, with the diameter of proximal end 42 progressively widening or expanding in diameter as it extends in the proximal direction, creating a gradual increase in the cross-sectional area of the distal tip portion 40 as it extends proximally. In some embodiments, and as illustrated, the flared or otherwise enlarged diameter proximal end 42 of the distal tip portion 40 is formed monolithically with the rest of the distal tip portion 40. However, in other instances, the flared or enlarged diameter region may be formed separately and attached to another portion of the distal tip portion 40. Among various advantages, the flared configuration of the proximal end 42 of the distal tip portion 40 allows for secured coupling between the distal tip portion 40 and a distal end of the blood pump 50. For instance, the flared configuration of the proximal end 42 of the distal tip portion 40 increases the bond strength between the distal tip portion 40 and the distally extending post 56 of the adapter 55, and subsequently the bond strength between the distal tip portion 40 and the blood pump 50. For example, in some embodiments, the bond strength between the distal tip portion 40 and the distally extending post 56 of the adapter 55 may be increased by a factor of 5 or more, by a factor of 8 or more, or by a factor of 10 or more, relative to a bond between the distally extending post 56 of the adapter 55 and a similar distal tip portion devoid of a braid.

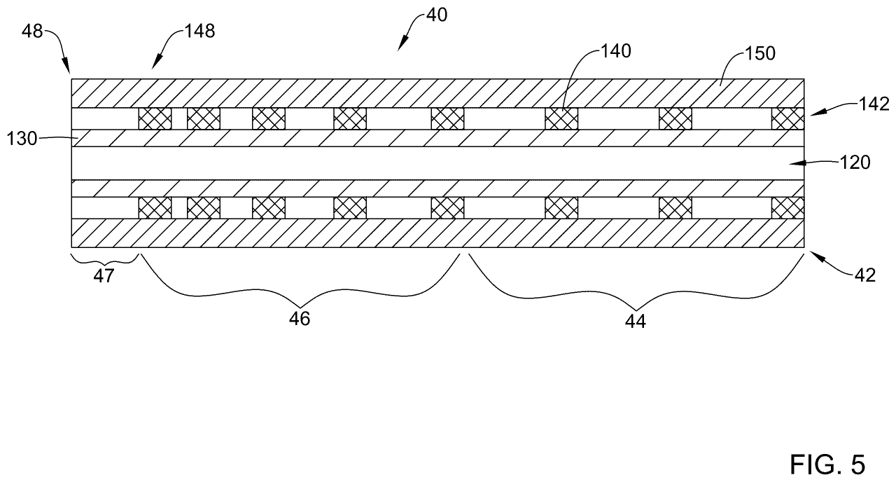

In some embodiments and as illustrated, the distal tip portion 40 may include multiple components or layers, including at least an outer layer 150, an inner layer 130, and a braided reinforcement layer 140. The braided reinforcement layer 140 may be positioned between the inner layer 130 and the outer layer 150. The braided reinforcement layer 140 may include a tubular braid extending along a length of the distal tip portion 40, including the proximal terminal region 44 and the distal terminal region 46. The tubular braid of the braided reinforcement layer 140 may extend along substantially an entire length of the distal tip portion 40, and may have a distal end 148 proximate the distal end 48 of the distal tip portion 40 and proximal end 142 proximate the proximal end 42 of the distal tip portion 40. The braid of the braided reinforcement layer 140 may extend along the flared proximal end of the distal tip portion 40 such that the braid of the braided reinforcement layer 140 surrounds the distally extending post 56 of the adapter 55. Further aspects of the braided reinforcement layer 140 will be discussed in greater detail in association with the description of FIG. 5.

The addition of the tubular braid of the braided reinforcement layer 140 to the distal tip portion 40 increases the strength and stiffness of the distal tip portion 40. In some embodiments, the addition of the tubular braid may increase the strength and stiffness by a factor of 1.5 or more, or 1.75 or more, relative to a similar distal tip portion devoid of a braid. Consequently, the distal tip portion 40 is better able to withstand forces acting upon the distal tip portion 40 or other components of the percutaneous circulatory support device. Furthermore, the addition of the braid 140 also increases axial strength, which facilitates positioning and supporting the cannula 30 in the left ventricle. The addition of the tubular braid of the braided reinforcement layer 140 may also significantly increase the bond strength between the distal tip portion 40 and the blood pump 50, such as, the bond strength between the distal tip portion 40 and distally extending post 56 of the adapter 55 secured to the cannula 30. This increase in bond strength may bring about more control in the positioning and support of the cannula 30 in the left ventricle, as well as greater durability and reliability in the operation and placement of the device. In some embodiments, the tubular braid of the braided reinforcement layer can increase the bond strength between the distal tip portion 40 and the post 56 of the adapter 55 by over 3 times, over 5 times, over 8 times, or 10 times in some instances. The effect on bond strength is influenced by the braid's diameter, the material composition of the braid, and its coupling mechanism to the blood pump 50, among other factors.

In some embodiments, the tubular braid of the braided reinforcement layer 140 extends the full length of the distal tip portion 40, from the proximal end 42 of the distal tip portion 40 to the distal end 48 of the distal tip portion 40. In some embodiments and as illustrated, a distalmost segment 47 of the distal terminal region 46 of the distal tip portion 40 is devoid of the braid, the distal end 148 of the tubular braid of the braided reinforcement layer 140 having terminated proximally to the distalmost segment 47 of the distal terminal region 46. In other words, the distal end 148 of the tubular braid of the braided reinforcement layer 140 may be located proximal of the distal end 48 of the distal tip portion 40. Consequently, the distalmost segment 47 of the distal terminal region 46 may be markedly more flexible in comparison to the braided portion of the distal tip portion 40 including the braided reinforcement layer 140. This configuration may facilitate atraumatic contact with the tissue and the ability to better absorb any forces acting on the distal tip portion 40 or other components of the percutaneous circulatory device.

FIG. 5 depicts a cross-sectional view of distal tip portion 40 of an illustrative percutaneous circulatory support device. In some embodiments and as illustrated, the distal tip portion 40 may include multiple components or layers, including at least an outer layer 150, an inner layer 130, and a braided reinforcement layer 140 therebetween. In some embodiments, the distal tip portion 40 also includes a lumen 120 extending longitudinally therethrough which is configured to accommodate a guidewire.

In some embodiments and as illustrated, the outer layer 150 is disposed outwardly of both the inner layer 130 and the tubular braid of the braided reinforcement layer 140. In other embodiments, the tubular braid of the braided reinforcement layer 140 is embedded into at least a portion of the outer layer 150 and/or embedded into at least a portion of the inner layer 130. In some embodiments, the outer layer 150 may be constructed from one or more polymers (for example, polyether block amide, thermoplastic polyurethane, or the like). In some instances, the outer layer 150 may be doped with or include a radiopaque filler material, or otherwise include a radiopaque material. In other embodiments, not illustrated, the outer layer 150 may be comprised of multiple sub-layers, with each sub-layer constructed out of one or more polymeric materials. For example, an outer layer may be composed of two sub-layers, three sub-layers, or the like.

The inner layer 130 may be constructed from one or more polymers, such as a lubricious polymer (for example, polytetrafluoroethylene), or other appropriate lubricious material known to a person of ordinary skill in the art. An inner surface of the inner layer 130 may define the lumen 120 extending through the distal tip portion 40 to receive a guidewire therethrough. When positioned within the lumen 120, the guidewire may straighten the curved shape of the distal terminal region 46 of distal tip portion 40 such as shown in FIG. 5.

In some embodiments and as illustrated, the tubular braid of the braided reinforcement layer 140 may be partially embedded into the outer layer 150 such that polymeric material of the outer layer 150 is disposed between filaments of the tubular braid and bonds to the outer surface of the inner layer 130. In other embodiments, a tie layer (not shown) may be disposed on the outer surface of the inner layer 130 to facilitate bonding with the outer layer 150.

The tubular braid of the braided reinforcement layer 140 may be constructed from one or more materials (i.e., a mixture) appropriate to the size and shape of the distal tip portion, compatibility with the material or composition of the inner layer 130 and/or outer layer 150 to which the braided reinforcement layer 140 is bonded to and/or embedded in, or desired customization of properties of the tubular braid. In some examples, the tubular braid of the braided reinforcement layer 140 is constructed out of a plurality of interwoven flat ribbon wires. In other examples, the tubular braid of the braided reinforcement layer 140 is constructed out of a plurality of interwoven round wires. In some examples, the wires forming the tubular braid of the braided reinforcement layer 140 may be constructed out of a radiopaque material, for example, to aid in fluoroscopic imaging. In some examples, the wires forming the tubular braid of the braided reinforcement layer 140 are constructed from a heat set nitinol wire, which may be chosen, for example, to aid in retaining the preconfigured curved shape of the distal tip portion 40. In some examples, a stiffer material may be chosen for better pushability, or a more flexible material may be chosen to enhance trackability and reduce the risk of vessel trauma. The material used to construct the braid may or may not uniform; the individual wires (e.g., strands or filaments) of the braided reinforcement layer 140 may be composed of differing materials. For example, the tubular braid of the braided reinforcement layer 140 may be formed to include one or more wires of radiopaque material and one or more wires of nitinol material to procure the fluoroscopic and shape-retention advantages associated with each, respectively. By choosing disparate materials to construct each individual wire of the braided reinforcement layer 140, the braided reinforcement layer 140, for example, may gain the thermal, mechanical, or electrical properties of each wire type into one combination.

Distal tip portion 40 is constructed to have a varying stiffness profile along its length. This stiffness profile may be influenced by the material selection discussed above. Additionally or alternatively, the varying stiffness profile may be attributable to the varying structure of the tubular braid of the braided reinforcement layer 140 along the length of the distal tip portion 40 from the distal end 48 to the proximal end 42. For instance, the distal end 148 of the braid may have a first per inch crosses (“pic”) count and the proximal end 142 may have a second pic count. The first pic count of the distal end 148 may be different from the second pic count of the proximal end 142. The first pic count of the distal end 148 may be greater than the second pic count of the proximal end 142, for instance, providing a stiffer proximal terminal region 44 than the distal terminal region 46 of the distal tip portion 40. In one example, the distal end 148 of the braid may have a pic count of about 200, while the proximal end 142 of the braid may have a pic count of about 20. The pic count may vary continuously or step-wise between the distal end 148 of the braid and the proximal end 142 of the braid. In other example, the distal end 148 of the braid may have a pic count in the range of 100 to 200 or more while the proximal end 142 of the braid may have a pic count in the range of 10 to 50. Other constructions are also contemplated. The exact pic count chosen for each respective end may be influenced by desired stiffness, flexibility requirements, shape of the distal end of the distal tip portion, bond strength requirements, and manufacturing constraints, among others.

As the tubular braid of the braided reinforcement layer 140 approaches the proximal end 42 of the distal tip portion 40, there may be a gradual transition in pic count from the first pic count at the distal end 148 of the braid to the second pic count at the proximal end 142 of the braid. In some embodiments and as illustrated, the first pic count is greater than the second pic count, such that the wires forming the braid become closer together as the braided wires approach the distal end of the distal tip portion 40. As a consequence, distal tip portion 40 becomes stiffer as it approaches the proximal end 42 of the distal tip portion 40.

This variable stiffness profile enables the distal tip portion 40 to gain the advantages of both a stiffer portion, for example the ability to withstand forces, facilitate positioning, or support the cannula within the left ventricle, and a flexible portion, for example atraumatic contact with the tissue and ability to absorb forces. The gradual transition between the greater first pic count at the distal end 148 of the tubular braid of the braided reinforcement layer 140 to the lesser second pic count of the tubular braid of the braided reinforcement layer 140 at the proximal end 142 of the tubular braid allows for a smoother stiffness profile, enhancing the overall trackability and maneuverability of the device and enhancing kink resistance by eliminating abrupt changes in flexibility. For similar reasons, the distal tip portion 40 is better able to maintain its shape when it has a smoother stiffness profile. When multiple materials are chosen to construct the braid, a gradual transition in stiffness ensures against the introduction of stress points along the distal tip portion 40 caused by changes in material. By mitigating the formation of stress points, the durability and ability to maintain the shape of the distal tip portion 40 is also enhanced.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device, comprising:
a blood pump including an inlet and an outlet;
an impeller disposed within a housing of the blood pump and being rotatable relative to the housing to cause blood to flow into the inlet, through the housing, and out of the outlet;
a motor operably coupled to the impeller, the motor configured to rotate the impeller relative to the housing; and
a distal tip portion coupled to the blood pump and extending distally therefrom, the distal tip portion including:
an inner layer;
an outer layer; and
a braided reinforcement layer including a braid disposed between the inner layer and the outer layer,
wherein the braid comprises a stiffness profile that varies a stiffness of the braid along a length of the distal tip portion.

2. The percutaneous circulatory support device of claim 1, wherein a distal end of the braid has a first pic count, and a proximal end of the braid has a second pic count, wherein the first pic count is different than the second pic count.

3. The percutaneous circulatory support device of claim 2, wherein the first pic count is greater than the second pic count, and wherein the braid is configured to provide a gradual transition in pic count along the length of the distal tip portion from the distal end of the braid to the proximal end of the braid.

4. The percutaneous circulatory support device of claim 2, wherein a ratio between the first pic count and the second pic count is 3:1 or more.

5. The percutaneous circulatory support device of claim 2, wherein the first pic count is in a range of 100 to 200, and the second pic count is in a range of 10 to 30.

6. The percutaneous circulatory support device of claim 1, wherein the distal tip portion has a flared proximal end configured to couple with a distal end of the blood pump.

7. The percutaneous circulatory support device of claim 6, further comprising an adapter configured to couple the distal end of the blood pump and the flared proximal end of the distal tip portion.

8. The percutaneous circulatory support device of claim 6, wherein the braid extends along the flared proximal end of the distal tip portion.

9. The percutaneous circulatory support device of claim 8, wherein the flared proximal end of the distal tip portion extends over and overlaps with the adapter.

10. The percutaneous circulatory support device of claim 1, wherein the distal tip portion comprises a distal terminal region devoid of the braid.

11. A percutaneous circulatory support device, comprising:
a blood pump including an inlet and an outlet;
an impeller disposed within a housing of the blood pump and being rotatable relative to the housing to cause blood to flow into the inlet, through the housing, and out of the outlet;
a motor operably coupled to the impeller, the motor configured to rotate the impeller relative to the housing;
an adapter at a distal end of the blood pump, the adapter including a distally extending post; and
a distal tip portion surrounding and coupled to the distally extending post of the adapter, the distal tip portion including:
an inner layer;
an outer layer; and
a braided reinforcement layer including a braid disposed between the inner layer and the outer layer;
wherein the braid comprises a varying pic count along a length of the braid, with a first pic count at a distal end of the braid and a second pic count at a proximal end of the braid.

12. The percutaneous circulatory support device of claim 11, wherein the first pic count is greater than the second pic count, and wherein the braid is configured to provide a gradual transition in pic count along the length of the braid from the distal end of the braid to the proximal end of the braid.

13. The percutaneous circulatory support device of claim 11, wherein the distal tip portion further comprises a flared proximal end.

14. The percutaneous circulatory support device of claim 13, wherein the braid extends along the flared proximal end of the distal tip portion.

15. The percutaneous circulatory support device of claim 13, wherein the flared proximal end of the distal tip portion surrounds the distally extending post of the adapter.

16. The percutaneous circulatory support device of claim 11, wherein the distal tip portion comprises a distal terminal region devoid of the braid.

17. A percutaneous circulatory support device, comprising:
a blood pump including an inlet and an outlet;
an impeller disposed within a housing of the blood pump and being rotatable relative to the housing to cause blood to flow into the inlet, through the housing, and out of the outlet;
a motor operably coupled to the impeller, the motor configured to rotate the impeller relative to the housing; and
a distal tip portion coupled to the blood pump and extending distally therefrom, the distal tip portion including:
an inner layer;
an outer layer; and
a braided reinforcement layer disposed between the inner layer and the outer layer;
wherein the braided reinforcement layer includes a braid having a varying pic count along a length of the braid, with a first pic count at a distal end of the braid and a second pic count at a proximal end of the braid,
wherein the second pic count is less than the first pic count and the braid is configured to provide a gradual transition in pic count along the length of the braid from the distal end of the braid to the proximal end of the braid.

18. The percutaneous circulatory support device of claim 17, wherein the distal tip portion further comprises a flared proximal end configured to couple with a distal end of the blood pump via an adapter, wherein the flared proximal end of the distal tip portion extends over and overlaps with a distally extending post of the adapter.

19. The percutaneous circulatory support device of claim 18, wherein the braid extends along the flared proximal end of the distal tip portion.

20. The percutaneous circulatory support device of claim 17, wherein the distal tip portion comprises a distal terminal region devoid of the braid.
